(19) United States
(12) Patent Application Publication
(10) Pub. No.: US 2017/0202668 A1
(43) Pub. Date: Jul. 20, 2017

(54) DEVICE FOR THE ENDOVASCULAR TREATMENT OF A CARDIAC VALVE IN VIEW OF A PERCUTANEOUS VALVE REPLACEMENT

(71) Applicant: Mustapha LADJALI, Rueil Malmaison (FR)

(72) Inventor: Mustapha LADJALI, Rueil Malmaison (FR)

(21) Appl. No.: 15/326,278

(22) PCT Filed: Jul. 13, 2015
(86) PCT No.: PCT/EP2015/066003

§ 371 Date: Jan. 13, 2017

(30) Foreign Application Priority Data
Jul. 15, 2014 (FR) 1456778

(51) Int. Cl. A61F 2/24; A61B 17/04

(52) U.S. Cl. CPC A61F 2/2445; A61F 2/2466; A61B 17/0487; A61F 2250/001; A61F 2220/0033; A61F 2250/0098; A61B 2017/0488; A61B 2017/0496

(57) ABSTRACT

The invention relates to a device for treating a native valve comprising a body having two elongated segments, each elongated segment having a connecting end, both connecting ends being able to be bound to each other, and a traction end, the traction end including an attachment element for attachment to a traction system. Each traction end comprises a locking element.

### CROSS-REFERENCE TO RELATED APPLICATIONS

This application is a National Stage of International Application No. PCT/EP2015/066003, filed Jul. 13, 2015. This application, in turn, claims priority to French Patent Application No. 1456778, filed Jul. 15, 2014. Each of which is incorporated herein by reference.

### FIELD OF THE INVENTION

The present invention relates to a treatment device for a valve including a body having two elongated segments, each elongated segment having:

a connecting end, both connecting ends being able to be interconnected, and

a traction end, the traction end including an attachment element of a traction system.

### BACKGROUND

This device is notably applied to the treatment of cardiac valves and in particular to the treatment of tricuspid valves.

The heart includes valves which are present at the input and at the output of the right ventricle (tricuspid and pulmonary valve) and of the left ventricle (mitral and aortic valve). These valves ensure a unique circulation of the blood flow, avoiding any blood reflux at the end of the ventricular contraction.

However diseases or deformities affect the proper operation of the valves. In particular, the latter may suffer from lesions thus allowing a reflow or regurgitation towards the ventricle or the auricle having expelled the blood flow. The problem of regurgitation leads to an abnormal dilation of the ventricle which, in the long run, produces heart failure.

A native valve treatment consists in replacing the native valve with a prosthesis.

It is known how to operate damaged tricuspid valves or mitral valves with an open heart, by sewing a prosthesis on the natural ring of the tricuspid valve or on the native ring of the mitral valve.

However, an open heart operation has many disadvantages, notably for elderly patients or affected with weakening pathologies.

On the other hand, the implantation of a prosthesis via a percutaneous route at the tricuspid valve or the mitral valve is not possible for at least the following reasons:

the tricuspid valve or the mitral valve cannot be viewed in fluoroscopy;

the valve ring has variable sizes from one patient to another, the required range of prosthesis is wide;

the tricuspid ring has significant distensibility, generating a risk of migration of the implanted prosthesis by dilation of the implantation area.

### SUMMARY

An object of the invention is to obtain a treatment device giving the possibility of implanting a prosthesis of a predefined size in a not very invasive way and with little perturbation of the operation of the valve when the device is applied.

For this purpose, the object of the invention is a device of the aforementioned type, characterized in that each traction end comprises a locking element.

The device according to the invention may comprise one or several of the several features, taken individually or according to all the technically possible combinations:

the device includes at least one closing ring, the closing ring being movable relatively to the traction ends between an unlocking position and a locking position, wherein the ring connects the traction ends with each other;

each elongated segment has a central region between the connecting end and the traction end, the central region of a segment is a flexible strip;

both elongated segments are separable;

each traction end comprises a locking element, each locking element being attached to the ring when the ring is in a locking position;

the ring comprises at least one eyelet, the traction end of each elongated segment being introduced into the eyelet in the locking position, the locking element being able to be attached in the eyelet;

the locking element comprises an abutment movable, relatively to the elongated segment, between an introduction position and a blocking position;

the ring comprises a diaphragm, the diaphragm being able to attach both traction ends; and

the connecting ends are permanently bound, the body having a loop shape.

The object of the invention is also a treatment kit comprising
a treatment device as defined above, and
a traction system able to tract the traction ends by reversibly being attached in the respective attachment elements.

The treatment kit according to the invention may comprise one or several of the following features, taken individually or according to all the technically possible combinations:

the kit comprises at least two guide-wires, each guide-wire being able to separately guide each elongated segment when the traction end is tracted by the traction system; and

the kit comprises a ring stake including at least one axial retention member of the ring able to allow a relative movement between the traction ends and the ring during the conveyance of the traction system.

### BRIEF DESCRIPTION OF THE DRAWINGS

The invention will be better understood upon reading the description which follows, only given as an example, and made with reference to the appended drawings, wherein:

FIG. 1 is a sectional view of a treatment kit according to the invention before a traction step;

FIG. 2 is a view similar to FIG. 1 of the treatment kit according to the invention subsequently to the traction step;

FIG. 3 is a view similar to FIG. 1 of the treatment kit according to the invention subsequently to the locking of the ring;

FIG. 4 is a view of the body of the treatment device of the treatment kit,

FIG. 5 is a view of the locking ring of the treatment device of the treatment kit,

FIG. 6 is a view similar to FIG. 1 of the treatment kit according to the invention, subsequently to deployment of the prosthesis,

FIG. 7 is a view similar to FIG. 4 of the body of the treatment device of another treatment kit according to the invention,

FIG. 8 is a side view of a further treatment kit according to the invention before a traction step, during the treatment of a mitral valve;

FIG. 9 is a top view of the treatment device of FIG. 8, set into place in a mitral valve,

FIG. 10 is a view similar to FIG. 9 of a treatment device of yet another treatment kit according to the invention set into place in a mitral valve.

### DETAILED DESCRIPTION

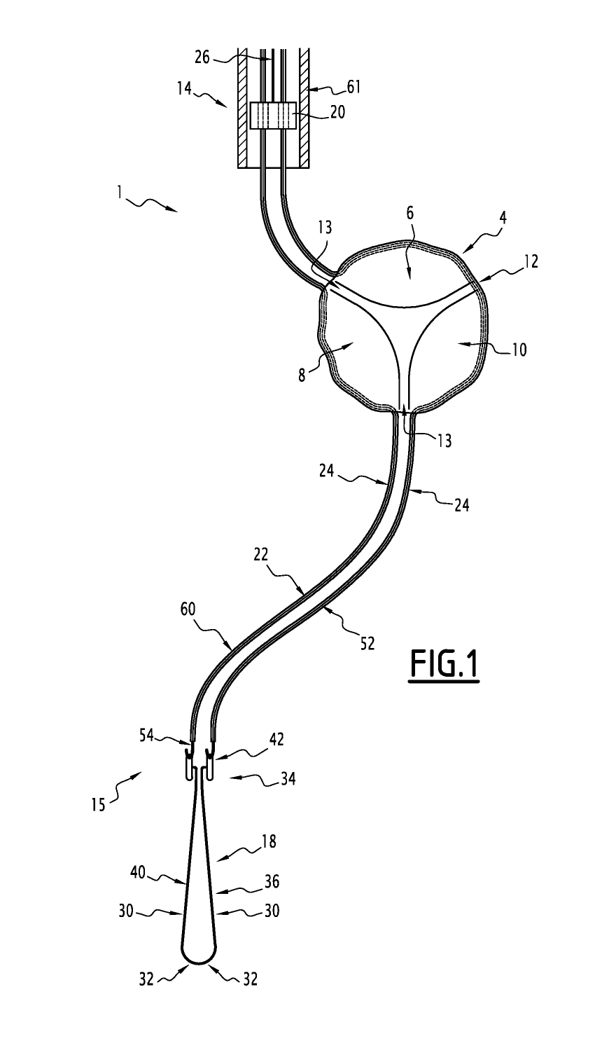

A first treatment kit 1 according to the invention is illustrated in FIGS. 1 to 6.

The first treatment kit 1 is intended for treating a valve, notably for treating a cardiac valve and in particular a tricuspid valve of a patient.

A tricuspid valve 4 is illustrated in FIG. 1. The tricuspid valve 4 is formed by an anterior sheet 6, a septal sheet 8 and a posterior sheet 10. The sheets 6, 8, 10 are attached on a native fibrous valve ring 12. Further, the sheets 6, 8, 10 are separated by commissures 13.

The tricuspid valve 4 is accessible through an upper approach 14 of the heart and through a lower approach 15 of the heart. The upper approach 14 of the heart is an access to the superior vena cava, approached through the jugular vein of the patient. The lower approach 15 of the heart is an access to the lower vena cava approached through the femoral vein in the thigh of the patient

The treatment kit 1 according to the invention is intended for the treatment of a tricuspid valve by setting into place a prosthesis.

The treatment kit 1 comprises a treatment device 16 including a body 18 and a closing ring 20 of the body. The treatment kit 1 further comprises a traction system 22 able to set into place the body 18 of the treatment device 16 in the tricuspid valve 4.

The treatment kit 1 further comprises at least two guide-wires 24 and a ring stake 26 facilitating the setting into place of the device 16.

The treatment kit 1 further comprises at least one prosthesis 72. Further, the treatment kit 1 comprises means for setting into place the prosthesis 72.

Advantageously, the treatment kit 1 comprises a plurality of prostheses 72 of different diameters.

The prosthesis 72 is for example a prosthetic valve intended to functionally replace the native valve. The prosthesis 72 is intended to be set into place in the valve ring 12.

The prosthesis 72 is advantageously tubular.

The outer diameter of the prosthesis 72 is for example comprised between 20 mm and 40 mm, advantageously between 25 mm and 33 mm.

For example, the diameter of the prosthesis is 27 mm or 29 mm.

In the treatment kit 1, the treatment device 16 is intended to form a closed loop after its setting into place. The loop forms a new prosthetic ring around native valve sheets 6, 8, 10 and close to the fibrous valve ring 12.

The treatment device 16 is intended for preparing a release site for a secondarily implanted prosthesis 72. This site may be located by fluoroscopy (and echography) and is calibrated, allowing the implantation of a prosthesis 72 of a reliably determined size. Finally, the device contributes to preventing periprosthetic leaks by flattening the native sheets 6, 8, 10 on the external surface of the prosthesis 72 to be implanted.

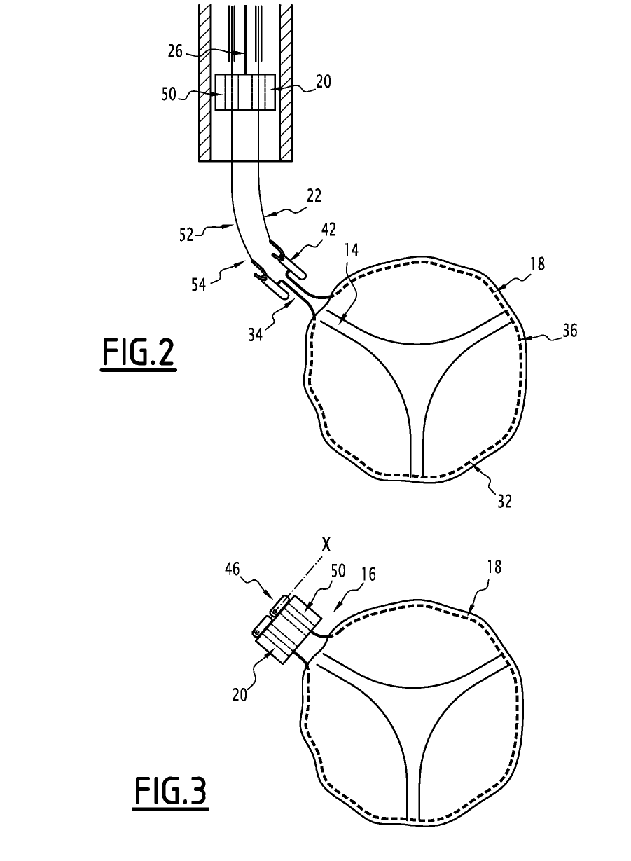

FIG. 1 and FIG. 2 illustrate the treatment device 16 before its setting into place. FIG. 3 illustrates the treatment device 16 at the end of its setting into place.

The body 18 is intended to form the inside of the loop, the loop being locked by the closing ring 20 in the treatment kit 1.

The body 18 has two elongated segments 30.

The length is adapted to the implantation of a cylindrical prosthesis 72 with a diameter comprised between 20 mm and 40 mm and advantageously between 25 mm and 33 mm.

Each elongated segment 30 substantially has the same length. Each elongated segment 30 is intended to be placed on a portion of the perimeter of the fibrous valve ring 12, on either side of the native valve.

Each elongated segment 30 has a connecting end 32, intended to connect both elongated segments 30 with each other, a traction end 34 intended for cooperating with the traction system 22 and a central region 36 located between the connecting end 32 and the traction end 34.

In this example, the connecting ends 32 are permanently bound, the body 18 having a loop shape. In other words, both segments 30 are permanently bound through their connecting ends 32.

The central region 36 of an elongated segment 30 consists of a flexible strip 40.

The width of the strip 40 is able to give the possibility of obtaining a stabilized orientation of the body 18 relatively to the valve ring 12 and relatively to the sheets 6, 8 10.

Further, the width of the strip 40 gives the possibility of obtaining a stabilized orientation of the prosthesis 72 relatively to the body 18 when the prosthesis 72 is set into place.

The width of the strip 40 is advantageously comprised between 1 mm and 8 mm, for example equal to 4 mm.

The strip for example consists of a metal material, advantageously nitinol, of a fabric for example of polyester or wires for example Goretex®, made to be radio-opaque.

The central region 36 of the body 18 is advantageously radio-opaque.

Each traction end 34 includes an element 42 for attaching a traction system 22.

In the example illustrated in FIGS. 1 to 4, the attachment element 42 consists of a hole allowing the capture of a hook.

Each traction end 34 further comprises a locking element 44. In the treatment kit 1, each locking element 44 is able to cooperate with the closing ring 20 as this will be described subsequently.

For example, the locking element 44 comprises a movable abutment 46, relatively to the elongated segment 30, between an introduction position and a blocking position.

The abutment 46 is of an elongated shape along a main axis X. The abutment 46 is movable in rotation around the traction end of the elongated segment 30.

The abutment 46 is illustrated in FIGS. 1, 2 and 4 in the introduction position and in FIG. 3 in the blocking position. In the introduction position, the main axis X of the abutment 46 is located in the extension of the axis of the elongated segment 30. In the blocking position, the main axis of the abutment 46 is perpendicular to the extension of the axis of the elongated segment 30.

The closing ring 20 is movable relatively to the traction ends 34 between an unlocking position and a locking position. In the locking position, the ring 20 permanently and irreversibly connects the traction ends 34 to each other.

The closing ring 20 comprises at least one eyelet 50, the traction end 34 of each elongated segment being introduced into the eyelet 50 in the locking position, the locking element being able to be attached in the eyelet 50.

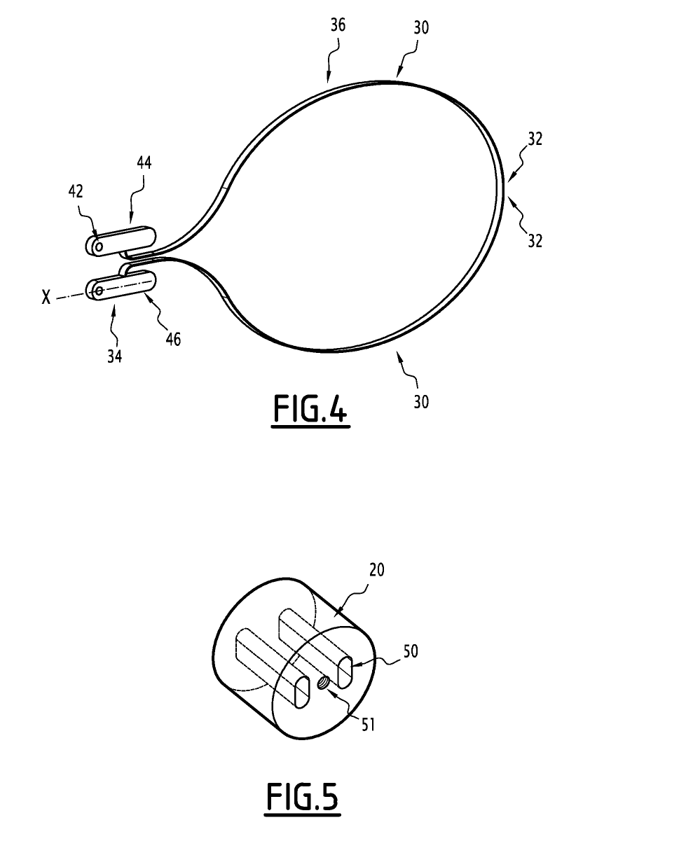

In the kit, the ring 20 illustrated in detail in FIG. 5, includes two eyelets 50, each being intended to cooperate with a traction end of one of the segments 30.

The eyelets 50 are able to cooperate with the abutments 46. The eyelets 50 are thus with a shape mating the abutments 46. When the abutments 46 are in the introduction position, they are able to be introduced into the eyelets 50. When the abutments 46 are in the blocking position, they cannot pass into the eyelets 50.

The ring 20 further includes a means 51 for attaching the stake of the ring 26. The attachment between the ring stake 26 and the ring 20 is reversible. For example, the ring stake 26 has a threaded end and the ring 51 has a hole with a mating threading.

The traction system 22 is intended to transport the body 18 from the lower approach 15 of a patient as far as the tricuspid valve 4 as this will be described subsequently.

The traction system 22 is able to tract the traction ends 34 by being reversibly attached in respective attachment elements 42.

The traction system 22 for example comprises two cables 52, including an attachment end 54 and a gripping end, each cable 52 being received into a sheath 60.

The cables 52 of the traction system 22 have a sufficient length so as to be drawn from the upper approach 14, from the outside of the patient, during the transport of the device 16 of the lower approach 15 as far as the tricuspid valve 4. For example, the length of the cables 52 of the traction system 22 is comprised between 50 cm and 2 m.

Each attachment end 54 is able to cooperate with the attachment element 42 of the traction end 34 of a segment 30. In the illustrated example, the attachment end includes a hook capable of gripping the attachment end 42 through the hole.

Each guide-wire 24 is able to separately guide each elongated segment 30 when the traction end 34 is tracted by the traction system 22. A first guide-wire 24 is intended to pass on one side of the native valve, and a second guide-wire 24 is intended to pass on an opposite side of the native valve.

Each sheath 60 has a sufficient diameter so as to be able to receive the guide-wire 24, the cable 52, and allow their sliding.

For example, the internal diameter of the sheaths is comprised between 1 mm and 3 mm.

The length of the guide-wires 24 is advantageously greater than the length of the cables 52 of the traction system 22. Notably, the length of the guide-wires is comprised between 2 m and 3 m, advantageously equal to 2 m 60 cm.

The area of the outer cross-section of each sheath 60 is less than the area of the inner cross-section of each eyelet 50. Thus, each sheath 60 receiving the cable 52 and the guide-wire 24 is able to slide in an eyelet 50.

The ring stake 26 is a tool for setting the ring into place. The ring stake 26 is able to cooperate with the attachment means 51 of the ring 20. The ring stake 26 is housed in an external introduction sheath 61.

The ring stake 26 includes at least one axial retention member of the ring 20 able to allow a relative movement between the traction ends 34 and the ring 20 during the conveyance of the traction system 22.

The operation of the treatment kit according to the invention, during the treatment of a tricuspid valve, will now be described with reference to FIGS. 1 to 6.

A treatment kit 1 is provided. An upper approach 14 of the heart is prepared. A lower approach 15 of the heart is prepared.

A first guide-wire 24 is introduced through the upper approach 14 into the commissure 13 between the anterior sheet 6 and the septal sheet 8 of the tricuspid valve 4. The first guide-wire 24 is placed under the septal sheet 8 so that it runs along the inside of the valve ring 12 and emerges through the commissure 13 between the septal sheet 8 and the posterior sheet 10.

The second guide-wire 24 is introduced through the upper approach 14 into the commissure 13 between the anterior sheet 6 and the septal sheet 8 of the tricuspid valve 4. The second guide-wire 24 is placed under the anterior sheet 6 and the posterior sheet 10 so that it runs along the inside of the valve ring 12 and emerges through the commissure 13 between the septal sheet 8 and the posterior sheet 10.

Both guide-wires 24 thus encircle the sheets 6, 8, 10 inside the valve ring 12.

A first end of each guide-wire 24 is recovered in the lower approach 15 while the second end of each guide-wire 24 remains positioned in the upper approach 14.

Both guide-wires 24 once set into place, thus form two separate guiding paths around the native ring.

The traction system 22 is introduced through the upper approach 14 with its attachment ends 54 directed downwards. Each sheath 60 is slipped on around a guide-wire 24 and is slid from the upper approach 14 to the lower approach 15.

Each cable 52 of the traction system 22 is deployed in a sheath 60 by following one of the guiding paths formed by one of the guide-wires 54, until the attachment ends 54 of the traction system emerge through the lower approach 15. For this, each cable 52 is deployed by sliding in the sheath 60.

The attachment ends 54 then protrude out of the sheaths 60 as illustrated in FIG. 1. The gripping ends of the traction system 22 protrude in the upper approach 14.

Both cables 52 of the traction system 22 thus encircle the sheets 6, 8, 10 inside the valve ring 12.

The traction ends 34 of the elongated segments 30 of the device 16 are then attached to the respective attachment ends 54 of the traction system 22 in the lower approach 15 as illustrated in FIG. 1.

In this example, each hook located at the attachment end 54 is introduced into a hole forming an attachment element 42 in a traction end 34.

The gripping ends of the traction system 22 are caught in the upper approach 14 and then are drawn by the operator. Each cable 52 of the traction system then slides in the sheath 60. Each cable 52 drives one of the elongated segments 30 because of the attachment of an attachment end 54 of the traction system 22 to an attachment element 42 of the body 18. The elongated segments 30 are thus each moved upwards along a respective guide-wire 24.

The elongated segments are thus moved away from each other. A first segment 30 is placed under the septal sheet 8 so that it runs along the inside of the valve ring 12 and emerges through the commissure 13 between the septal sheet 8 and the posterior sheet 10. A second segment 30 is placed under the anterior sheet 6 and the posterior sheet 10 so that it runs along the inside of the valve ring 12 and emerges through the commissure 13 between the septal sheet 8 and the posterior sheet 10. The traction ends 34 protrude out of the valve 4.

The ring 20 is set into place in the upper approach 14 by sliding the cables 52 of the traction system 22 and the guide-wires 24 in the eyelets 50 of the ring 20.

When the segments 30 are in place, the operator positions the ring 20 in the vicinity of the traction ends 34 through the upper approach 14 by means of the stake 26. The operator maintains the traction ends 34 in place by means of the cables 52.

The locking elements 44 are introduced into the eyelets 50 of the ring 20.

The abutment 46 is drawn into its introduction position so as to protrude out of the eyelets 50 of the ring 20 after the introduction. The abutment 46 is displaced towards its blocking position. The ring 20 is placed in a locking position so as to secure both traction ends 34.

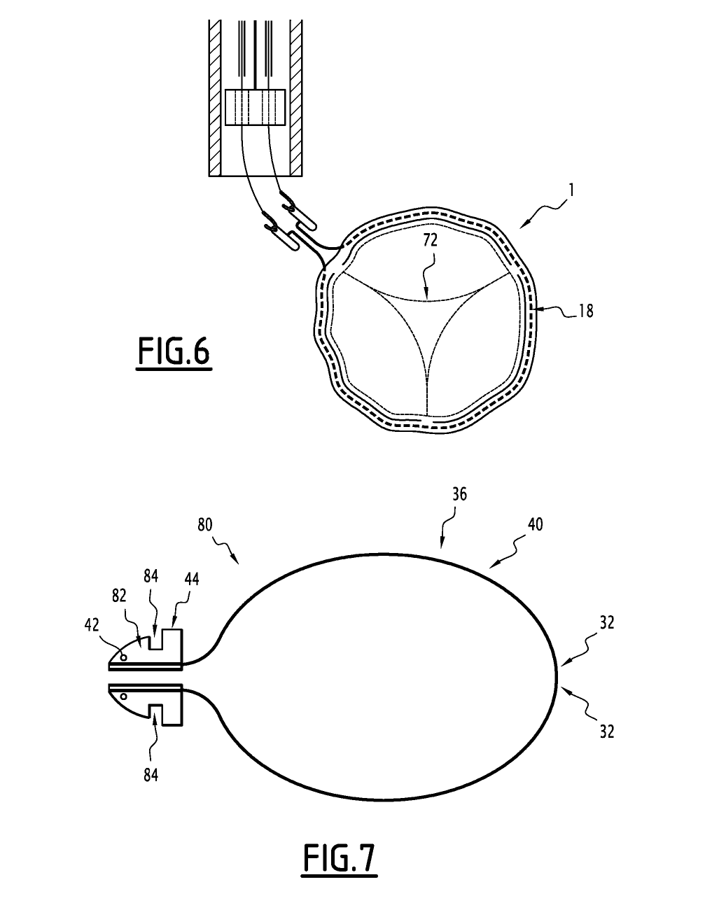

After the step for setting into place the body 18 illustrated in FIG. 2, a step for setting into place the prosthesis 72 is illustrated in FIG. 6. The setting into place of the prosthesis 72 is carried out before unhooking the traction system and removing the guide-wires. The locking by the ring 20 illustrated in FIG. 3 is advantageously achieved before setting the prosthesis 72 into place.

The treatment device 16 illustrated in FIG. 3 according to the invention gives the possibility of preparing the setting into place of the prosthesis 72 illustrated in FIG. 6 and limits the risks of a leak during the setting into place of the prosthesis 72.

Advantageously, the treatment kit 1 comprises a plurality of prostheses 72 of different diameters.

Once the body 18 of the treatment device 16 has been set in position, the operator selects the most adapted prosthesis 72. A step for viewing the area encircled by the body 18 according to the invention gives the possibility of determining the size of the prosthesis 72 to be placed and of thus selecting the prosthesis 72 having the best characteristics from among the plurality of prosthesis 72.

The prosthesis 72 is introduced into the valve ring 12 through the lower approach by means for setting into place which are not shown. The prosthesis 72 is deployed. The prosthesis 72 is retained in position by the body 18 which encircles it inside the native valve ring 12.

The operator unhooks the attachment ends 54 of the traction system 22 from the traction ends 34.

The operator removes the guide-wires 24 through the upper approach 14. The operator removes the ring stake 26 through the upper approach 14. Thus, only the device 16 remains positioned in the heart.

Advantageously, the different steps are carried out under echographic and fluoroscopic control.

The treatment kit according to the invention therefore gives the possibility of achieving a circling of the valve 4 from the inside of the valve 4. Further, the ring 20 which maintains the body 18 closed is on the outside of the valve 4, it thereby remains accessible.

A second treatment kit 80 is illustrated in FIG. 7. The second treatment kit 80 differs from the treatment kit 1 in that the locking elements 44 consist of a snap-fastening module 82 and the ring is able to cooperate with the snap-fastening module 82.

For example, the snap-fastening module 82 comprises notches 84 intended for receiving the ring.

A third treatment kit according to the invention differs from the treatment kits described earlier, in that both elongated segments 30 may be separated. Further, each connecting end 32 includes an element for attachment to the other connecting end 32.

The elongated segments 30 for example have different lengths according to the position which they are intended to have in the valve 4. For example, the elongated segment 30 intended to be positioned under the septal sheet 8 is shorter than the elongated segment 30 intended to be positioned under the anterior sheet 6 and under the posterior sheet 10.

The operation of the third treatment kit according to the invention differs in that the connecting ends 32 are attached to each other at the lower approach 15 after the setting into place of the central regions 34 of the elongated segments 30 in the valve ring 12.

A fourth treatment kit according to the invention differs from the treatment kits described earlier in that a connecting end 32 includes an attachment member. The attachment member is able to allow traction of the body 18 of the device 16 in a direction opposite to the direction of traction by the traction system 22.

The operation of the fourth kit according to the invention differs from the operation of the kits described earlier in that the body 18 is tracted by the traction system 22 towards the upper approach 14 and by means of the attachment member towards the lower approach 15 in order to have a specific setting into place of the body in the valve ring 12.

The attachment member comprises a traction wire, and for example two eyelets secured to the body 18. The traction wire is removable i.e. it is movable between an attachment position in which it is secured to the body 18 and a release position in which it is withdrawn from the body 18. For example, in the attachment position, the traction wire passes into both eyelets, the surgeon may pull on both ends of the traction wire at the same time in order to act on the position of the connecting end 32 and therefore on the position of the body 18. When the surgeon wants to remove the wire, he/she calls on one of the two ends of the traction wire, the wire then slides through the eyelets and is released from the body 18.

In an embodiment not shown, the central region 34 is a thick wire. Many other locking systems may also be contemplated.

For example, the closing ring 20 includes a safety pin able to be attached with a ring of the locking element 44.

Alternatively, the ring 20 is a sheath as a key ring consisting of two hoops able to clasp the locking elements 44.

Alternatively, the locking system is activated by means of the ring stake 26.

Alternatively, the ring 20 comprises a diaphragm, the diaphragm being able to be attached to both traction ends 34. The diaphragm has an adjustable internal diameter by means of the ring stake 26 between a first diameter allowing the introduction of the traction ends 34 and a second diameter allowing the locking of the locking elements 44 in the ring 20.

Alternatively, the ring 20 is a flat strip notably in tissue. Its thickness is much smaller than its other dimensions and is for example less than 3 mm.

Each element described above is non-traumatic. For example, the attachment elements include rounded ends in order not to be sharp. Alternatively, the attachment elements include spiked ends but remain covered by a sheath as long as they are not in the desired position.

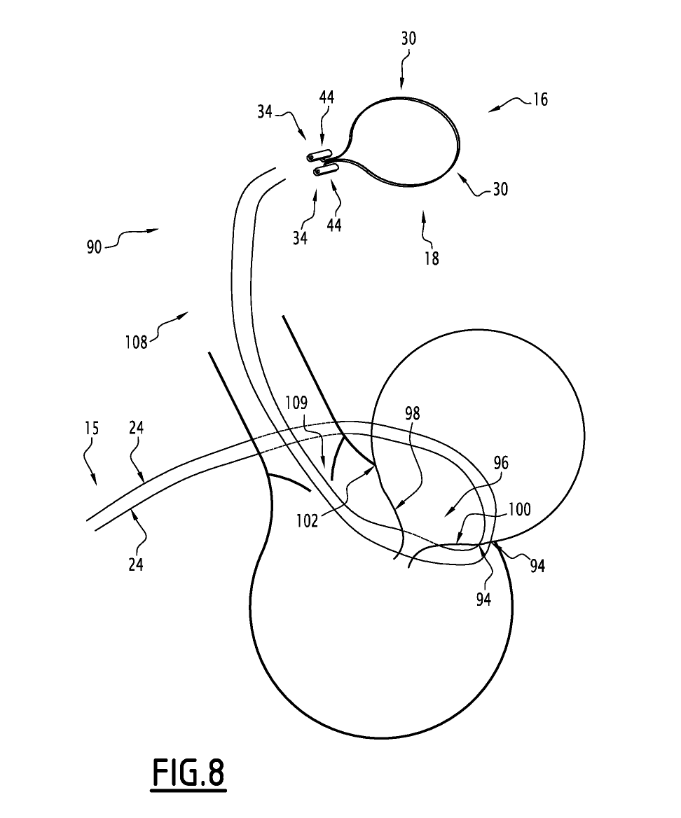

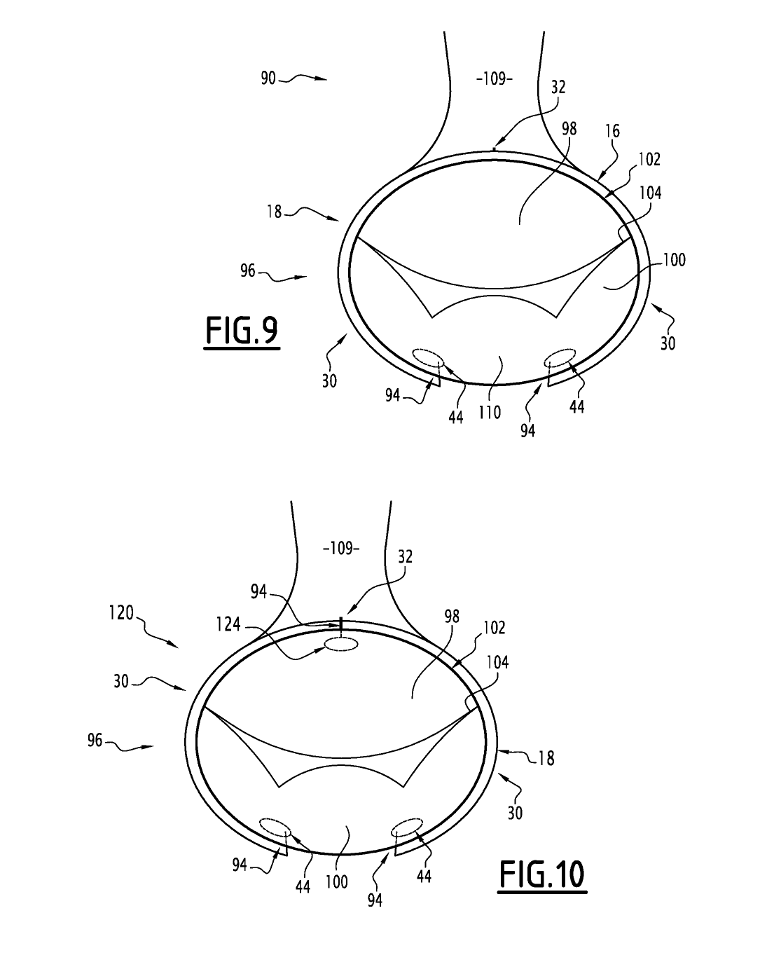

A fifth treatment kit 90 is illustrated in FIGS. 8 and 9.

The fifth treatment kit 90 differs from the treatment kits 1, 80 described earlier in that each locking element 44 is able to anchor a segment 30 to a tissue in an attachment point 94. For example, the locking element 44 is able to be attached in a native valve ring or in a native valve tissue.

The anchoring of each segment 30 to the tissue is achieved by transfixation between a first face of the tissue and a second face of the tissue at the attachment point 94. I.e. at the level of the attachment point 94, a portion of the segment 30 is on one side of the tissue and another portion of the segment 30 is on the other side of the tissue on the side of the second face.

The attachment point 94 of a first traction end 34 is for example at a distance from the attachment point 94 of the other traction end 34 so that once the treatment device 16 is set into place, the traction ends 34 are at a distance from each other. The treatment device 16 thus forms an open loop after its setting into place.

Advantageously, the locking element 44 includes an abutment 46 as described earlier. When the abutments 46 are in the introduction position, they are able to cross the tissue. When the abutments 46 are in the blocking position, they cannot pass through the tissue. Advantageously, the abutments 46 are needles.

In the illustrated example, the fifth treatment kit 90 is intended for treating a mitral valve 96 of a patient. A mitral valve 96 is illustrated in FIG. 8 and FIG. 9. The mitral valve 96 includes an anterior sheet 98 and a posterior sheet 100, attached on a native valve ring 102 and separated by a commissure 104.

The treatment device 16 is intended to be set into place under the valve sheets 98, 100 at most near the ring 102. The treatment device 16 is intended to be attached to the ring 102 by means of the locking elements 44 at attachment points 94 by transfixation. The loop formed by the device 16 surrounds the valve sheets 98, 100 at most close to the native valve ring 102 of the mitral valve 94. In the illustrated example, the locking elements 44 of traction ends 34 are intended to be attached in the tissue, on either side of the central festoon 110 of the posterior sheet 100 of the mitral valve 96.

The attachment of the body 18 to the tissue by two attachment points 94 by means of the locking elements 44 reinforces the maintaining of the body 18 as close as possible to the native valve ring 102. In the example illustrated in FIGS. 8 and 9, the loop formed by the treatment device 16 forms a new prosthetic ring around valve sheets 98, 100 of the mitral valve 96 as close as possible to the valve ring 102 in the chamber for purging the left ventricle.

The operation of the fifth treatment kit 90 according to the invention, during the treatment of a mitral valve 94 will now be described with reference to FIGS. 8 and 9. The operation differs from the operation described earlier for the treatment of a tricuspid valve 4 at least in that the path defined by the guide-wires 24 and then by the sheaths 60 is different. The fifth treatment kit 90 is provided. A lower approach 15 through the femoral vein of the heart and an approach via a trans-aortic route 108 through the femoral artery giving access to the heart through the aorta 109 are prepared.

A first guide-wire 24 is introduced through the lower approach 15 as far as the right auricle and then crosses the inter-auricular septum, arrives in the left auricle and crosses the native mitral ring 102 on the side of the posterior sheet 100 of the mitral valve 96. The first guide-wire 24 is placed under the posterior valve sheet 100 so that it runs along the inside of the native mitral ring 102 of the mitral valve 96 and emerges through the approach of the femoral artery via a trans-aortic route 108.

The second guide-wire 24 is introduced through the lower approach 15 as far as the right auricle and then, crosses the inter-auricular septum, arrives in the left auricle and crosses the native mitral ring 102 on the side of the posterior sheet 100 of the mitral valve 96 at a distance, for example, at a distance comprised between 1 mm and 15 mm and advantageously between 1 mm and 10 mm, of the first guide-wire 24. The second guide-wire 24 is placed under the posterior valve sheet 100 and makes its way in the direction opposite to that of the first guide-wire 24, so that it runs along the inside of the native mitral ring 102 of the mitral valve 96 and emerges through the approach of the femoral artery via a trans-aortic route 108.

Both guide-wires thus encircle the valve sheets 98 and 100 as close as possible to the native mitral ring 102 of the mitral valve 96.

A first end of each guide-wire 24 is recovered in the trans-aortic approach 108 by the femoral artery while the second end of each guide-wire 24 remains positioned in the lower approach 15. Both guide-wires 24 once they are set into place thereby form two separate guiding paths.

As earlier, each sheath 60 is slipped on around a guide-wire 24. Each cable 52 of the traction system 22 is deployed in a sheath 60 by following one of the guiding paths formed by one of the guide-wires 24, until the attachment ends 54 of the traction system 22 emerge through the trans-aortic approach 108 and the femoral artery. The traction ends 34 of the elongated segments 30 of the device 16 are then attached to the respective attachment ends 54 of the traction system 22 in the trans-aortic approach 108, through the femoral artery.

The gripping ends of the traction system 22 are caught in the lower approach 15 by the femoral vein and then drawn by the operator. The elongated segments 30 are thus moved up each along a respective guide-wire 24. The elongated segments 30 are thus moved away from each other. A first segment 30 passes on one side around valve sheets 98, 100 and attain the native valve ring 102 or the native valve tissue of the posterior sheet 100 through an attachment point 94. A second segment 30 passes on the other side around valve sheets 98, 100 and attains the native valve ring 102 or the native valve tissue of the posterior sheet 100 through a second attachment point 94 at a distance from the attachment point 94 of the first segment 30. The second attachment point 94 is, for example, located at a distance comprised between 1 mm and 15 mm and advantageously between 1 mm and 10 mm of the first attachment point 94.

The locking elements 44 are introduced into the ring 102 or the tissue. The abutment 46 is drawn into its introduction position so as to protrude on the other side of the ring 102 or of the tissue after introduction. Next, the abutment 46 is displaced towards its blocking position. The tissue secures both traction ends 34.

After the step for setting the body 18 into place, a step for setting into place the prosthesis 72 is carried out. The operator then attaches the attachment ends 54 of the traction system 22 of the traction ends 34. The operator removes the guide-wires 24.

The treatment device 16 of the fifth treatment kit 90 contributes to preventing periprosthetic leaks by securing the prosthesis 72 to be implanted not only to the valve sheets 98, 100, but also to the native valve ring 102 by tissue transfixation.

Further, the arrangement of the treatment device 16 in an open loop avoids that the surgeon has to perform a complete turn of the structure for implanting the prosthesis 72, and facilitates the setting into place of the prosthesis 72.

Alternatively, a locking ring 20 is added to the locking ends 44 after their crossing of the tissue at the attachment points 94. For example, the locking ring 20 is a strip placed above the native valve tissue or the native valve ring 102 and crossed by the locking elements 44. The locking elements 44 are therefore both attached to the tissue and to the ring 20. The locking ring 20 ensures maintaining the ring 102 leak-proof between both attachment points 94, for example if the tissue was torn between both attachment points 94. The loop formed by the body 18 is closed facing the central festoon 110 by the locking ring 20.

In an alternative, an upper approach 14 through the jugular vein is prepared, the guide-wires 24 are introduced from the upper approach 14 through the jugular vein and the gripping ends of the traction system 22 are caught in the upper approach 14.

A sixth treatment kit 120 is illustrated in FIG. 10. The sixth treatment kit 120 differs from the fifth treatment kit 90 in that the treatment device 16 has both the two locking elements 44 and an attachment member including an anchoring element 124. Like for the fourth treatment kit, the attachment member is on a connecting end 32. Further, the attachment member is able to allow traction of the body 18 of the device in a direction opposite to the traction direction by the traction system 22, by means of a wire maneuverable from the outside of the patient, from the trans-aortic approach 108 through the femoral artery.

The anchoring element 124 also gives the possibility of attaching the body 18 of the treatment device 16 to the tissue. The anchoring element 124 comprises a needle which is attached to the native valve tissue or to the native valve ring.

The body 18 set into place is maintained at the native valve tissue or at the native valve ring by three attachment points 94. The attachment point 94 of the anchoring element 124 is located on the valve substantially diametrically opposite to the attachment points 94 of the locking elements 44. Advantageously, the anchoring element 124 gives the possibility of attaching the device at the mitro-aortic continuity, i.e. in the continuity between the ring 102 of the mitral valve and the valve of the aorta 109.

The anchoring element 124 of this device allows the body 18 not to fall and to remain stable upon deploying the prosthesis 72 by traction on the wire. During heart contractions, the anchoring element 124 maintains stable strapping as close as possible to the ring 102 while allowing proper deployment of the prosthesis 72.

The anchoring element 124 allows a coaxial positioning of the prosthesis 72 relatively to the chamber for purging the right ventricle. The anchoring element 124 gives the possibility of attaching the native valve tissue around the treatment device 16 and the prosthesis 72 in order to avoid perivalvular leaks.

In an alternative, the attachment member further includes a clamping device. The clamping device is able to adjust the length of the body 18 in order to tighten the body 18 around the prosthesis 72.

The invention therefore gives the possibility of obtaining a treatment device 16 allowing preparation of a valve in a not very invasive way upon implanting a prosthesis and not perturbing much the operation of the valve when the device is set into place.

The treatment device 16 according to the invention is intended for a treatment via an endovascular route, i.e. that its setting into place is accomplished by passing through the inside of a blood vessel, and does not require any open heart operation.

The attachment of the locking ends 44 in the tissue and/or in the locking ring gives the possibility of maintaining the treatment device 16 relatively to the valve in its position selected by the surgeon upon its setting into place.

1. A treatment device for a native valve comprising a body having two elongated segments, wherein each elongated segment comprises:
a connecting end, each connecting end being able to be connected to another connecting end, and
a traction end, wherein the traction end comprises an attachment element for attachment of a traction system, wherein each traction end comprises a locking element.

2. The treatment device according to claim 1, wherein each elongated segment has a central region between the connecting end and the traction end, the central region of a segment is a flexible strip.

3. The treatment device according to claim 1, wherein both elongated segments are separable.

4. The treatment device according to claim 1, wherein the treatment device comprises at least one closing ring, the closing ring being movable relatively to the traction ends between an unlocking position and a locking position, wherein the ring binds the traction ends with each other.

5. The treatment device according to claim 4, wherein each locking element is attached to the ring when the ring is in the locking position.

6. The treatment device according to claim 4, wherein the ring comprises at least one eyelet, the traction end of each elongated segment being introduced into the eyelet in the locking position, the locking element being able to be attached in the eyelet.

7. The treatment device according to claim 4, wherein the ring comprises a diaphragm, the diaphragm being able to attach both traction ends.

8. The treatment device according to claim 1, wherein the locking element comprises an abutment movable, relatively to the elongated segment, between an introduction position and a blocking position.

9. The treatment device according claim 1, comprising an attachment member connected to the body away from each traction end, the attachment member including a removable traction wire.

10. The treatment device according to claim 9, wherein the attachment member is provided with an anchoring element in the tissue.

11. A treatment kit comprising:
a treatment device according to claim 1, and
a traction system able to tract the traction ends by reversibly being attached in respective attachment elements.

12. The treatment kit according to claim 11 comprising:
at least two guide-wires (24), each guide-wire being able to separately guide each elongated segment when the traction end is tracted by the traction system.

13. The treatment kit according to claim 11 comprising a treatment device, wherein the treatment device comprises at least one closing ring, the closing ring being movable relatively to the traction ends between an unlocking position and a locking position, wherein the ring binds the traction ends with each other, and comprising a ring stake comprising at least one axial retention member for the ring able to allow a relative movement between the traction ends and the ring during conveyance of the traction system.
